(19) United States
(12) Patent Application Publication
(10) Pub. No.: US 2018/0140513 A1
(43) Pub. Date: May 24, 2018

(54) Skin Cover and Medical Device

(71) Applicant: Geana Jones, Nipomo, CA (US)

(72) Inventor: Geana Jones, Nipomo, CA (US)

(21) Appl. No.: 15/571,776

(22) PCT Filed: Jun. 2, 2016
(86) PCT No.: PCT/US16/35544

§ 371 Date: Nov. 3, 2017

Related U.S. Application Data
(63) Continuation-in-part of application No. 14/704,235, filed on May 5, 2015, now abandoned.

(51) Int. Cl. A61K 8/02; A61K 9/70; A61Q 1/14; A61Q 1/00; A61K 47/34; A61K 8/891; A61Q 1/02

(52) U.S. Cl. CPC A61K 8/0208; A61K 9/7076; A61Q 1/145; A61K 2800/42; A61K 47/34; A61K 8/891; A61Q 1/02; A61Q 1/00

(57) ABSTRACT

A skin cover is provided for covering of tattoos, scars, and other similar blemishes. The skin cover having at least two layers, a covering layer and an adhesive layer and the skin covering having transparent and translucent features for quickly and easily covering blemishes and to seamlessly blend into a user's skin.

### PRIOR HISTORY

This patent application is a U.S. National Stage (371) patent application that claims the benefit of or priority to pending international application no. PCT/US16/35544 filed 2 Jun. 2016 and claims benefit to U.S. application Ser. No. 14/704,235 filed in the United States Patent and Trademark Office on May 5, 2015.

### FIELD OF THE INVENTION

The present invention generally relates to the field of skin treatment devices, and more particularly to a tattoo cover and medical cover assembly for enhancing the skin and coving unwanted blemishes.

### BACKGROUND

Tattoos are a permanent or semi-permanent coloring or discoloring artistic expression on the skin. The permanent tattoo is a well know art form dating back to the origins of mankind, and are traditionally applied by inserting ink or other coloring into the skin using needles, syringes, etc.

However, the artistic expression in tattoos often loses its desirability after a certain amount of time has passed. Oftentimes tattoos become embarrassing to the wearer, or can fade and become unattractive. In addition, other skin imperfections such as birth marks, scars, or other blemishes can exist on the skin of an individual and are needed to be covered or permanently corrected. Traditionally, processes for removing scarring and tattoos are unreliable, or require the user to undergo expensive, painful, and time consuming surgical procedures. Occasionally, scar tissue can form as a result of such procedures, and they require covering after the fact. Other methods for concealing scar and tattoos include liquid or gel concealers, however they have downsides such as smearing, staining clothing, and lack of durability.

Others utilize temporary tattoos or adhesive bandages to cover scars and other blemishes. However the traditional implements of these devices are typically unable to last for long periods of time, and often do not seamlessly blend into the user's skin. Thus, an improved device for covering such imperfections is greatly needed.

Various products exist on the market that attempt to solve these problems, but often involve problems of their own. For example, various cosmetics or makeup are often utilized on their own, or in conjunction with coverings. However, all such implementations all suffer from a similar flaw of being susceptible to sweat, athletic activity, or simply being rinsed off the skin in water (such as while swimming). Moreover, the problem of staining clothing exists with water-resistant cosmetics requiring dry cleaning, or permanently damaging the article.

Thus, there exists a need for a temporary patch that can apply to an individual's tattoo or skin imperfection that is easily applied, inexpensive, and long lasting.

### BRIEF SUMMARY

The present invention, therefore contemplates an improved, thin, self-adhesive soft skin covering. The purpose of which is to provide a new thin general purpose soft cover or covering to solve the above mentioned problems and allow a user freedom in deciding where and how to cover skin imperfections. A skin cover with the following indications is therefore provided.

Preferably, a skin covering according to the present invention will be thin, or ultra-thin and have a matter or semi gloss finish that comes in varying shades to match a user's skin tone and can be applied to the user's skin to block or hide existing tattoos or skin blemishes. Therefore the cover of the present invention is more efficient at concealing a permanent tattoo then previous skin covers.

In the preferred embodiment the skin cover is capable of blocking the visual and textural imperfections from being read through the top, and provides a sweat, abrasion, and waterproof covering that is unlikely to be wiped off or rinsed off by the user. In some embodiments the cover is transparent, translucent, or opaque, and in other embodiments it may preferably be available in several colors, shades, and tones. The preferred embodiments of the present invention contemplate that the skin cover will be soft and/or flexible and capable of conforming to the body as the user moves.

Preferably, in certain embodiments, the current invention contemplates being self adhesive or adhesive to the skin of the user. In certain embodiments it is contemplated to be semi-permanent in nature, in other embodiments the user can easily remove and reapply the skin cover without pain or irritation to the skin. Another advantage of certain embodiments is during long-term affixation to the skin, the present invention does not irritate the skin, an improvement over the prior art. In many ways this allows for a greater ease of use.

In one embodiment the current invention contemplates a skin cover for covering of blemishes comprising a first, semitransparent layer capable of preventing see through of the blemishes, a second, colored layer, the colored layer capable of being one or more skin tones or shades, and a third, adhesive layer, the adhesive layer affixed to the semitransparent layer, the adhesive layer being washable and reusable after being applied to a user's skin.

Such embodiments may preferably be augmented in certain ways, by way of example the skin cover may preferably comprise a peelable fourth layer affixed to the adhesive layer, the peelable layer capable of being peeled from and re-applied to the adhesive layer and capable of reducing wear and deposition of contaminants onto the adhesive layer. The skin cover has rounded, tapered, edges. The skin cover the skin cover is water and sweat resistant, and whereby the skin cover is capable of remaining affixed to the skin for extended periods when submerged in water. The first layer comprises five-to-twenty (5-20) durometer medical grade silicone rubber, and the third layer comprises a reusable silicone gel. The skin cover is between 0.018 and 0.022 inches in thickness. Or further comprising a medical salve incorporated into the third, adhesive layer, the salve capable of increasing the speed of healing of an underlying blemish. A centrally located absorbent layer affixed to the third adhesive layer, and the absorbent layer covering less than the entirety of the adhesive layer. A peelable layer affixed to the adhesive layer and in contact with the absorbent layer, the peelable layer capable of being peeled from and re-applied to the adhesive layer and capable of reducing wear and deposition of contaminants onto the adhesive and absorbent layers. A medical dopant applied to the absorbent layer, wherein the medical creates an environment of accelerated healing underneath the skin cover when applied to the skin.

In another embodiment the invention contemplates an ultra-thin, self-adhesive, washable skin covering comprising: a top portion, wherein the top portion is capable of blending seamlessly a user's skin, and a bottom portion, wherein the bottom portion contains an adhesive capable of selectively binding the skin covering to the user's skin.

Preferably in certain embodiments the top layer comprises five-to-twenty (5-20) durometer medical grade silicone rubber, and the bottom layer comprises a reusable silicone gel. The top layer of the skin covering comprises tapered edges. A peelable layer affixed to the bottom layer, the peelable layer capable of being peeled from and re-applied to the bottom layer and capable of reducing wear and deposition of contaminants onto the bottom layer. An absorbent patch centrally located and affixed to the bottom layer, the absorbent patch being smaller than the bottom layer, and the absorbent patch being compressed between the bottom layer and the peelable layer.

Other features and objects of the present invention will become more evident from a consideration of the following brief descriptions of patent drawings submitted in support of these specifications.

### DESCRIPTION OF THE DRAWINGS

The present invention may be better understood, and its numerous objects, features, and advantages made apparent to those skilled in the art by referencing the accompanying drawings.

FIG. No. 1(a) is a side on perspective of a tattooed neck and the skin cover according to the present invention.

FIG. No. 1(b) is a side on perspective of a tattooed neck with the skin cover according to the present invention applied to said neck.

FIG. No. 1(c) is a side on perspective of a tattooed neck with the skin cover according to the present invention applied to said neck, demonstrating the blending qualities of the skin cover.

FIG. No. 2(a) is a side on perspective of the skin cover of the present invention.

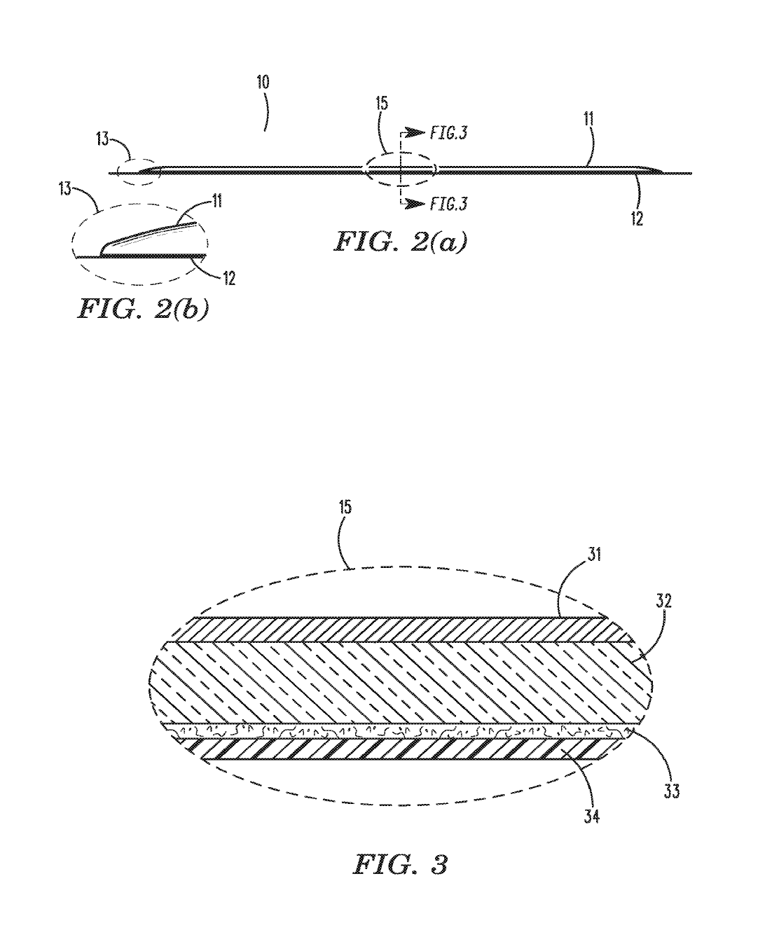

FIG. No. 2(b) is a side on perspective of a close up view of the edge of the skin cover of FIG. 2(a).

FIG. No. 3 is a side on, close up view of the skin cover of FIG. 2(a).

FIG. No. 4 is a perspective view of a preferred embodiment of the skin cover according to the present invention with backing.

FIG. No. 5 is a perspective view of a preferred embodiment of the skin cover according to the present invention being actively peeled from its backing.

FIG. No. 6 is a top view of a preferred embodiment of the skin cover according to the present invention.

FIG. No. 7 is a perspective view of a second preferred embodiment of the skin cover according to the present invention.

### DETAILED DESCRIPTION

As indicated above, the skin cover and medical device according to the present invention was designed, in part, to create a supplemental or primary skin covering that would enable the user to better control their body and how things are shown or healed. State of the art skin covers are cumbersome, oily, and do not provide the novel features of the present device as recited herein.

As stated, a primary objective of the skin covering system and medical device according to the present invention is to provide a covering 10 that will adequately cover a portion of the user's skin 20, particularly a tattooed or blemished portion 21. Top portion 11 of the medical skin patch is intended to blend seamlessly with skin 20, while bottom portion 12 preferably contains an adhesive that will selectively bind the patch 10 to skin 20 and the blemished portion 21 of the skin.

In a first preferred embodiment, show in greater specificity in FIG. 2. A basic skin cover 10 is preferably made using a top portion 11 five-to-twenty (5-20) durometer medical grade silicone rubber. The silicone is preferably mated with a reusable silicone gel 12 or reusable adhesive. Such adhesives preferably may be used multiple times over the life of the patch 10, or may preferably also be re-applicable. In certain embodiments, gel 12 contains more than one layer one, or more of which may preferably contain an active medical ingredient to aid in healing of the blemished area 21.

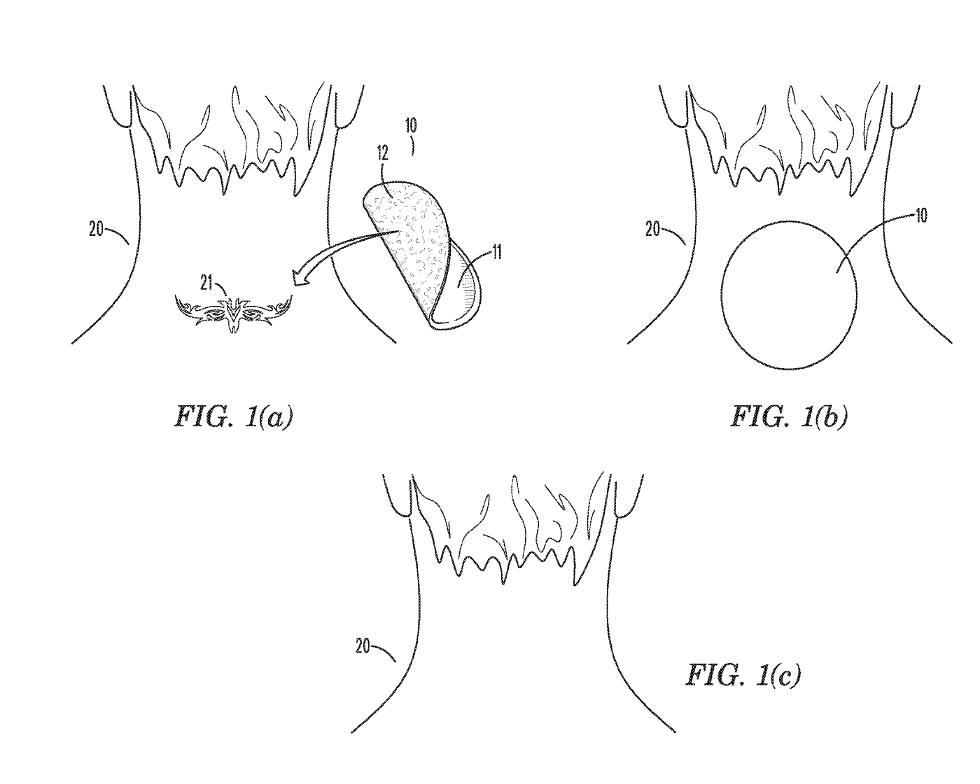

Looking with greater specificity to FIG. 2(b) edge portion 12 is preferably tapered and mates the edges of top 11 and bottom 12 portions of the patch such that there is a seamless transition not visible to the naked eye from a distance (as shown in FIG. 1(c)). This also allows the skin covering 10 to be manufactured in a more economical process, and such patches can be formed from multiple layers that are combined as desired. In certain preferred embodiments, patch 10 will be capable of being formed in a variety of shades or colors to more easily fade into the user's skin and may be made in a variety of shapes and sizes to more adequately accommodate the user's desired place of use. For example, a user may desire a smaller patch for a small ankle tattoo, but a larger patch to cover a tattoo on the thigh. Furthermore, the preferred adhesive 12 is waterproof and/or water resistant such that when a user sweats or is in water the patch does not wear off exposing the embarrassing blemish. This adhesive, therefore has increased longevity when compared to other covering methods.

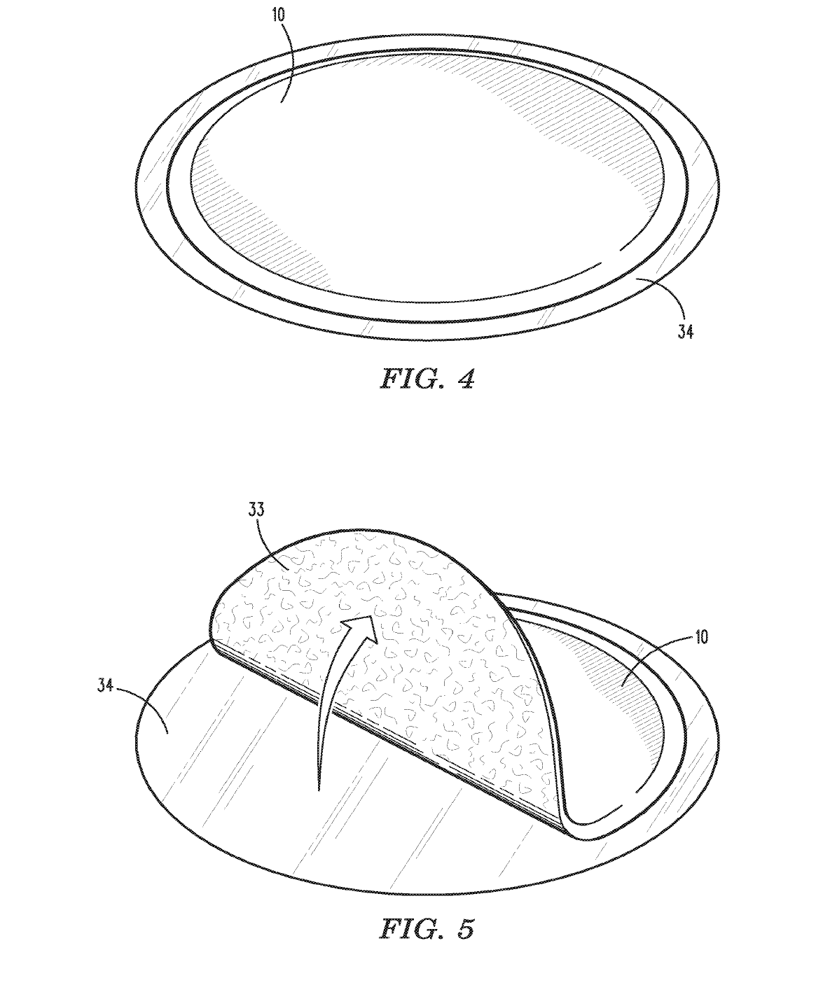

Looking now to FIG. 3 a preferred embodiment of a cross section of patch 10, carved out of middle portion 15 is show. In such embodiments the patch will contain several layers. Top portion 31 is cosmetic in nature and is connected to silicone body 32, and may be integrally formed therefrom. Cosmetic portion 31 may preferably be formed of a matte material such as SPI D3 or equivalent finish. Silicone body 32 is preferably manufactured from medical grade silicone, preferably between 0.018 and 0.022 inches in thickness, and tapering to between 0.004 and 0.006 in thickness at the edges. Adhesive 33 is then attached to the opposite side of the skin patch and may be used to connect a cover 34 to portions of the patch. Adhesive 33 is typically a medical grade silicone adhesive or tacky gel approximately 0.005 inches in thickness and applied to the majority or the entirety of the underside of body 32. Cover 34 is shown in greater detail in FIGS. 4 and 5 of the specification. In particular patch 10 is connected to the covering patch which prevents contamination of the adhesive 33 with dirt, grime, oils, or other contaminants. Then, as shown in FIG. 5 the patch 10 can easily be peeled from cover 34 thus exposing the adhesive 33. In many embodiments the adhesive 33 is washable using water or another solvent and thus cover 34 can be reused many times after uses and the patch 10 can then be re-peeled off of the cover and be re-applied to the skin.

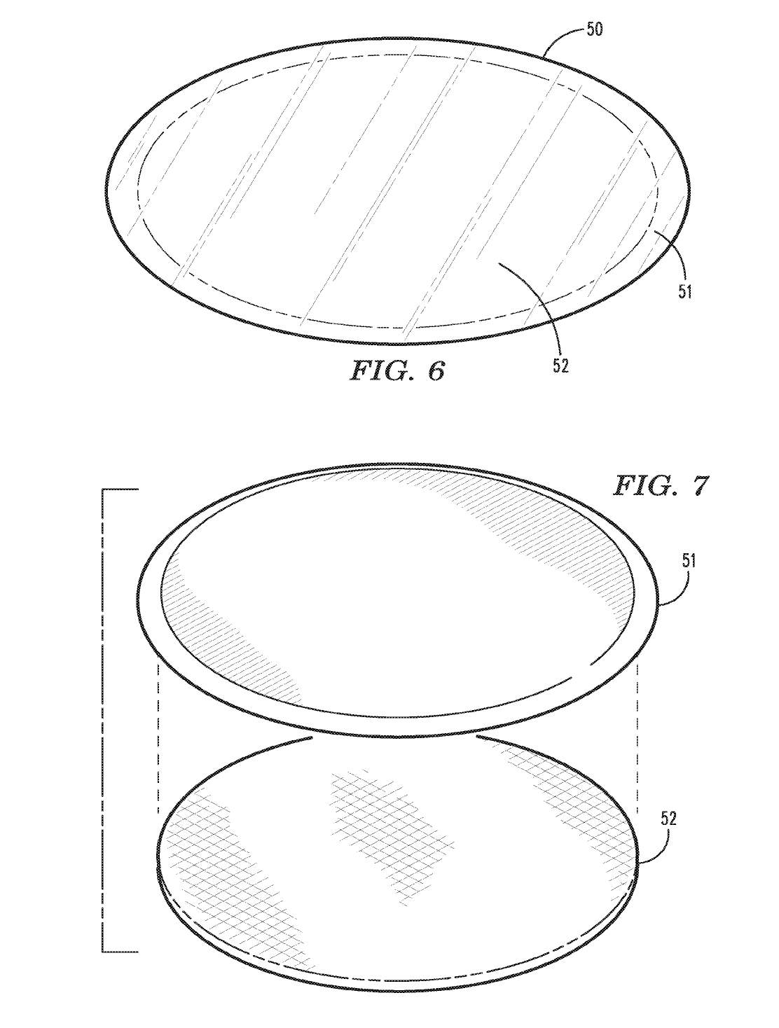

Looking now to FIGS. 6 and 7 another embodiment of the skin cover according to the present invention is shown. In this embodiment, skin cover 50 has adhesive portions 51 and absorbent portion 52. The absorbent portion or patch 52 has both advantages and disadvantages in the present invention. In particular, the absorbent patch may reduce the surface area of the adhesive portion 51 causing the patch to be likely to fall off, in addition, certain embodiments of the patch are transparent or translucent in nature, and absorbent patches may interfere with those properties, as well as the re-usability of the patch. However, patch 52 also preferably contains several advantages including the ability to contain a medical liquid or gel for treatment of blemishes or scarring. In addition such a patch may be preferably configured to absorb discharges from a blemish on the skin, and may preferably be replaceable in certain embodiments, thus greatly increasing the life of the skin patch 50 under certain conditions.

Manufacture of skin cover 10 follows a set of basic procedures to form the novel skin cover. First the liquid patch materials (silicon chains, having an alkene group) are mixed with pigment. The mixture comprises siloxanes and silicones being dimethyl, vinyl group-terminated combined with Silanamine, 1,1,1-trimethyl-N-(trimethylsilyl)-. Then the mixture is placed under vacuum and put into molds. The material in the mold is cured, at 302° F. and adhesive is applied to the flat side of the patch 10. The adhesive is then cured and a backing material is applied (as is commonly done with adhesive stickers). The adhesive comprises siloxanes and silicones, dimethyl beingmono(vinyl group)-terminated and cured with a platinum catalyst cured 45 minutes at 135° C. (275° F.). Teflon is commonly used as the backing.

Accordingly, although the invention has been described by reference to certain preferred and alternative embodiments, it is not intended that the novel arrangements be limited thereby, but that modifications thereof are intended to be included as falling within the broad scope and spirit of the foregoing disclosures, and the appended claims and drawings.

I claim:

1. A skin cover for covering of blemishes comprising:
a first, semitransparent layer capable of preventing see through of the blemishes;
a second, colored layer, the colored layer capable of being one or more skin tones or shades; and
a third, adhesive layer, the adhesive layer affixed to the semitransparent layer, the adhesive layer being washable and reusable after being applied to a user's skin.

2. The skin cover of claim 1 further comprising:
a peelable fourth layer affixed to the adhesive layer, the peelable layer capable of being peeled from and re-applied to the adhesive layer and capable of reducing wear and deposition of contaminants onto the adhesive layer.

3. The skin cover of claim 1 wherein:
the skin cover has rounded, tapered, edges.

4. The skin cover of claim 1 wherein:
the skin cover is water and sweat resistant, and whereby the skin cover is capable of remaining affixed to the skin for extended periods when submerged in water.

5. The skin cover of claim 1 wherein:
the first layer comprises five-to-twenty (5-20) durometer medical grade silicone rubber; and the third layer comprises a reusable silicone gel.

6. The skin cover of claim 1 wherein:
the skin cover is between 0.018 and 0.022 inches in thickness.

7. The skin cover of claim 1 further comprising:
a medical salve incorporated into the third, adhesive layer, the salve capable of increasing the speed of healing of an underlying blemish.

8. The skin cover of claim 1 further comprising:
a centrally located absorbent layer affixed to the third adhesive layer;
the absorbent layer covering less than the entirety of the adhesive layer.

9. The skin cover of claim 8 further comprising:
a peelable layer affixed to the adhesive layer and in contact with the absorbent layer, the peelable layer capable of being peeled from and re-applied to the adhesive layer and capable of reducing wear and deposition of contaminants onto the adhesive and absorbent layers.

10. The skin cover of claim 9 further comprising:
a medical dopant applied to the absorbent layer, wherein the medical creates an environment of accelerated healing underneath the skin cover when applied to the skin.

11. An ultra-thin, self-adhesive, washable skin covering comprising:
a top portion, wherein the top portion is capable of blending seamlessly a user's skin; and
a bottom portion, wherein the bottom portion contains an adhesive capable of selectively binding the skin covering to the user's skin.

12. The skin covering of claim 11 wherein:
the top layer comprises five-to-twenty (5-20) durometer medical grade silicone rubber;
and the bottom layer comprises a reusable silicone gel.

13. The skin covering of claim 12 wherein:
the top layer of the skin covering comprises tapered edges.

14. The skin covering of claim 13 further comprising:
a peelable layer affixed to the bottom layer, the peelable layer capable of being peeled from and re-applied to the bottom layer and capable of reducing wear and deposition of contaminants onto the bottom layer.

15. The skin covering of claim 14 further comprising:
an absorbent patch centrally located and affixed to the bottom layer;
the absorbent patch being smaller than the bottom layer; and
the absorbent patch being compressed between the bottom layer and the peelable layer.
